(19) United States
(12) Patent Application Publication
(10) Pub. No.: US 2025/0166170 A1
(43) Pub. Date: May 22, 2025

(54) MACHINE LEARNING BASED MEDICAL IMAGING ANALYSIS USING FEW SHOT LEARNING WITH TASK INSTRUCTIONS

(71) Applicant: Siemens Healthineers AG, Forchheim (DE)

(72) Inventors: Bogdan Georgescu, Princeton, NJ (US); Awais Mansoor, Potomac, MD (US); Sasa Grbic, Plainsboro, NJ (US); Dorin Comaniciu, Princeton, NJ (US)

(21) Appl. No.: 18/512,237

(22) Filed: Nov. 17, 2023

(51) Int. Cl. G06T 7/00; G06F 40/40

(52) U.S. Cl. CPC G06T 7/0012; G06F 40/40; G06T 2207/20081; G06T 2207/20092; G06T 2207/30004

(57) ABSTRACT

Systems and methods for performing a medical imaging analysis task using a machine learning based task network based on task instructions are provided. One or more input medical images of a patient and task instructions for performing a medical imaging analysis task are received. The one or more input medical images are encoded into imaging features using an image encoder network. The task instructions are encoded into text features using a text encoder network. The medical imaging analysis task is performed based on the imaging features and the text features using a machine learning based task network. Results of the medical imaging analysis task are output.

### TECHNICAL FIELD

The present invention relates generally to medical imaging analysis, and in particular to machine learning based medical imaging analysis using few shot learning with task instructions.

### BACKGROUND

Machine learning models have recently been proposed for performing various medical imaging analysis task, such as, e.g., segmentation, registration, classification, detection, diagnosis, etc. Typically, such machine learning models are trained for performing a specific medical imaging analysis task using supervised learning. Supervised learning requires a large amount of task specific medical images with expert annotations. However, acquiring such a large amount of annotated medical images is time-intensive, costly, and subject to variance between annotators.

### BRIEF SUMMARY OF THE INVENTION

In accordance with one or more embodiments, systems and methods for performing a medical imaging analysis task using a machine learning based task network based on task instructions are provided. One or more input medical images of a patient and task instructions for performing a medical imaging analysis task are received. The one or more input medical images are encoded into imaging features using an image encoder network. The task instructions are encoded into text features using a text encoder network. The medical imaging analysis task is performed based on the imaging features and the text features using a machine learning based task network. Results of the medical imaging analysis task are output.

In one embodiment, the task instructions comprise references to image regions in at least one of the one or more input medical images. The task instructions may comprise anatomical knowledge and task knowledge. The task knowledge may comprise at least one of a description of an anatomical abnormality, how the anatomical abnormality is represented, and how the anatomical abnormality can be detected in the one or more input medical images. The task instructions may be user-defined.

In one embodiment, the text features and the imaging features are aligned in a same latent space.

In one embodiment, the machine learning based task network is trained with self-supervised learning based on unannotated training medical images and text. In one embodiment, the machine learning based task network is trained with few shot learning using annotated training medical images and annotated task descriptions. In one embodiment, the machine learning based task network comprises an LLM (large language model) based task network.

In one embodiment, text-based medical data of the patient is received. The task instructions and the text-based medical data are encoded into the text features using the text encoder network.

In accordance with one or more embodiments, systems and methods for training a machine learning based task network for performing a medical imaging analysis task based on task instructions are provided. One or more training medical images and training task instructions for performing a medical imaging analysis task are received. The one or more training medical images are encoded into imaging features using a pretrained image encoder network. The training task instructions are encoded into text features using a pretrained text encoder network. A machine learning based task network is trained for performing the medical imaging analysis task based on the imaging features and the text features. The trained machine learning based task network is output.

In one embodiment, the machine learning based task network is trained with self-supervised learning based on unannotated training medical images and text. In one embodiment, the machine learning based task network is trained with few shot learning using annotated training medical images and annotated task descriptions.

These and other advantages of the invention will be apparent to those of ordinary skill in the art by reference to the following detailed description and the accompanying drawings.

### BRIEF DESCRIPTION OF THE DRAWINGS

FIG. 1 shows a method for performing a medical imaging analysis task using a machine learning model based on task instructions, in accordance with one or more embodiments;

FIG. 2 shows exemplary task instructions for detecting pneumothorax in chest x-ray images, in accordance with one or more embodiments;

FIG. 3 shows a method for training a machine learning based task network for performing a medical imaging analysis task based on training task instructions, in accordance with one or more embodiments;

FIG. 4 shows a workflow for training a machine learning based task network for performing a medical imaging analysis task based on training task instructions, in accordance with one or more embodiments;

FIG. 5 shows an exemplary artificial neural network that may be used to implement one or more embodiments;

FIG. 6 shows a convolutional neural network that may be used to implement one or more embodiments;

FIG. 7 shows a schematic structure of a recurrent machine learning model that may be used to implement one or more embodiments; and

FIG. 8 shows a high-level block diagram of a computer that may be used to implement one or more embodiments.

### DETAILED DESCRIPTION

The present invention generally relates to methods and systems for machine learning based medical imaging analysis using few shot learning with task instructions. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system. Further, reference herein to pixels of an image may refer equally to voxels of an image and vice versa.

Conventional machine learning models are trained for performing medical imaging analysis tasks via supervised learning using a large amount of annotated medical images. Such a large amount of annotated medical images is needed in supervised learning to implicitly encode all details and possible variations of performing the medical imaging analysis tasks through the examples.

In according with embodiments described herein, machine learning models for performing medical imaging analysis tasks are trained using few shot learning with task instructions. The task instructions provide a “recipe” on how the medical imaging analysis tasks are to be performed. By encoding the task instructions and medical images, the text encoding of the task instructions is used for task reasoning, thus overcoming the complexity of scaling the amount of annotated medical images needed for supervised learning, as well as using more contextual information provided by the task instructions.

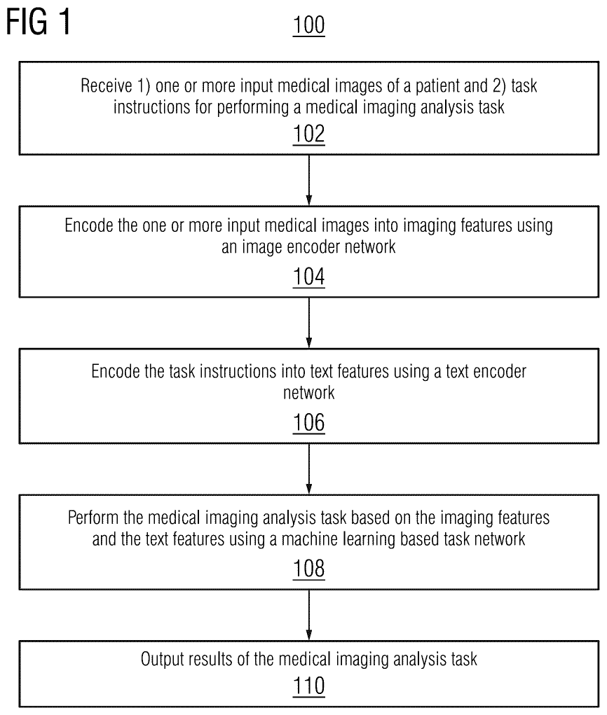

FIG. 1 shows a method 100 for performing a medical imaging analysis task using a machine learning model based on task instructions, in accordance with one or more embodiments. The steps of method 100 may be performed by one or more suitable computing devices, such as, e.g., computer 902 of FIG. 9.

At step 102 of FIG. 1, 1) one or more input medical images of a patient and 2) task instructions for performing a medical imaging analysis task are received.

The one or more input medical images may depict any anatomical object of interest of the patient, such as, e.g., organs, vessels, tumors, abnormalities, etc. The one or more input medical images may be of any suitable modality or modalities, such as, e.g., CT (computed tomography), MRI (magnetic resonance imaging), US (ultrasound), x-ray, or any other medical imaging modality or combinations of medical imaging modalities. The one or more input medical images may comprise 2D (two dimensional) images and/or 3D (three dimensional) volumes.

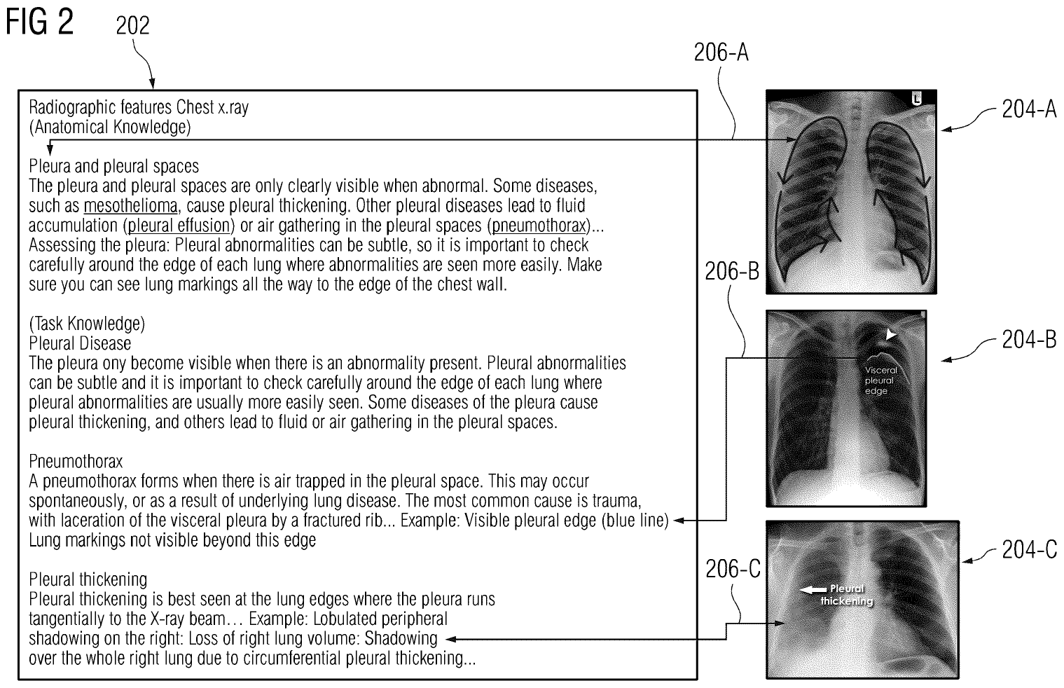

The task instructions act as a “recipe” or instructions on how the medical imaging analysis task is to be performed. The task instructions may comprise anatomical knowledge, task knowledge, and/or any other suitable information that may be utilized for performing the medical imaging analysis task. In one embodiment, the task instructions are associated with the one or more input medical images. For example, the task instructions may comprise explicit groundings or references to anatomical image regions and/or task specific image regions in at least one of the one or more input medical images. In one embodiment, the task instructions are user-defined task instructions generated by a user. However, the task instructions may be generated using any other suitable approach. Exemplary task instructions are shown in FIG. 2. The task knowledge may comprise, e.g., a description of anatomical abnormality, how the anatomical abnormality is represented, how the anatomical abnormality can be detected in the one or more input medical images, etc.

FIG. 2 shows exemplary task instructions 202 for detecting pneumothorax in chest x-ray images, in accordance with one or more embodiments. As shown in FIG. 2, task instructions 202 comprises anatomical knowledge and task knowledge. The anatomical knowledge describes anatomical knowledge of the pleura and pleural spaces. The task knowledge describes the radiological features of pleural disease, pneumothorax, and pleural thickening. Task instructions 202 comprise groundings or references 206-A, 206-B, and 206-C (collectively referred to as groundings 206) to anatomical image regions and/or task specific image regions in input medical images 204-A, 204-B, and 204-C (collectively referred to as input medical images 204). In particular, grounding 206-A grounds or references the pleura and pleural spaces described in task instructions 202 with anatomical regions of the pleura and pleural spaces shown in input medical image 204-A. Grounding 206-B grounds or references the radiological features of pneumothorax (“visible pleural edge” and “lung markings not visible beyond this edge”) in task instructions 202 with anatomical regions shown in input medical image 204-B. Grounding 206-C grounds or references the radiological features of pleural thickening (“shadowing over the whole right lung”) in task instructions 202 with anatomical regions shown in input medical image 204-C. In one example, input medical images 204 and task instructions 202 are the one or more input medical images and the task instructions received at step 102 of FIG. 1, respectively.

Referring back FIG. 1, in one embodiment, non-imaging medical data of the patient may also be received at step 102. Such non-imaging medical data may include, for example, text-based medical data, such as, e.g., radiology reports, laboratory reports, medical records, indications for imaging/examination, demographic information, administrative data, etc.

The one or more input medical images and the task instructions (and the non-imaging medical data) can be received by, for example, loading the input medical images, task instructions, and/or non-imaging medical data from a storage or memory of one or more computer systems and/or receiving the input medical images, task instructions, and/or non-imaging medical data from one or more remote computer systems. Such computer systems may comprise an EHR (electronic health record), EMR (electronic medical record), PHR (personal health record), HIS (health information system), RIS (radiology information system), PACS (picture archiving and communication system), LIMS (laboratory information management system), or any other suitable database or system. In some embodiments, the input medical images may be received directly from an image acquisition device, such as, e.g., a CT scanner, as the medical images are acquired.

At step 104 of FIG. 1, the one or more input medical images are encoded into imaging features using an image encoder network. The image encoder network may be machine learning based image encoder network, such as, e.g., an autoencoder, a VAE (variational autoencoder), or may be implemented according to any other suitable machine learning based architecture. The image encoder network receives the one or more input medical images as input and generates the imaging features as output. The imaging features are embeddings representing low-dimensional, dense vector representations of the relatively higher-dimensional input medical images in a latent space. The image encoder network is pretrained during a prior offline or training stage using a large set of training images. Once trained, the image encoder network is applied during an online or inference stage, for example, to perform step 104 of FIG. 1.

At step 106 of FIG. 1, the task instructions are encoded into text features using a text encoder network. In one embodiment, where non-imaging medical data of the patient is also received at step 102 of FIG. 1, the non-imaging medical data (e.g., the text-based medical data) is encoded with the task instructions into the imaging features using the image encoder network.

In one embodiment, the text encoder network is an LLM (large language model) based text encoder network. However, the text encoder network may be implemented according to any suitable machine learning based architecture. The LLM based text encoder network may be any suitable pre-trained deep learning based LLM. For example, the LLM based text encoder network may be based on the transformer architecture, which uses a self-attention mechanism to capture long-range dependencies in text. One example of a transformer-based architecture is GPT (generative pre-training transformer), which has a multilayer transformer decoder architecture that may be pretrained to optimize the next token prediction task and then fine-tuned with labelled data for various downstream tasks. GPT-based LLMs may be trained using reinforcement learning with human feedback for performing various natural language processing tasks. Other exemplary transformer-based architectures include BLOOM (BigScience Large Open-science Open-access Multilingual Language Model) and BERT (Bidirectional Encoder Representations from Transfers).

In one embodiment, the LLM based text encoder network is constrained to a specific medical domain. For example, the LLM based text encoder network may be constrained for the use case for detecting pneumothorax in chest x-ray images. To constrain the LLM based text encoder network, the LLM based text encoder network may be updated (e.g., trained, retrained, or fine-tuned) using, e.g., clinical data and data extracted from medical images using AI-based systems. Such extracted data may include, e.g., clinical measurements (e.g., diameters, volumes, distances, etc.), anatomical locations, detections, etc.

The LLM based text encoder network receives the task instructions (and possibly the non-imaging medical data) as input (e.g., as one or more prompts) and generates the text features as output. In one embodiment, the task instructions include specific prompting for the LLM based text encoder network (and possibly the LLM based task network utilized at step 108 of FIG. 1) to perform the task. Alternatively, at least some of the task instructions can be separately/directly input to the machine learning based task network (utilized at step 108 of FIG. 1). The text features are embeddings representing low-dimensional, dense vector representations of the relatively higher-dimensional task instructions (and possibly the non-imaging medical data) in the latent space. The LLM based text encoder network is pretrained during a prior offline or training stage using a large set of training text-based data. Once trained, the LLM based text encoder network is applied during an online or inference stage, for example, to perform step 106 of FIG. 1.

The text features and the image features are represented in the same latent space. The latent space may be refined to align corresponding image features and text features, such that similar features represent similar concepts. In one embodiment, the text and image features can be aligned in the latent space by pre-training the image encoder network and text encoder network on image-text pairs (such as, e.g., corresponding image-reports or other semantically annotated image/image region-text pairs). Pre-training can be performed by, e.g., minimizing the distance in the latent space between corresponding pairs and maximizing the distance between non-corresponding pairs. By aligning the image features and the text features, reasoning may be performed in the same latent feature space with interchangeable features (i.e., from either the text features or the image features).

At step 108 of FIG. 1, the medical imaging analysis task is performed based on the imaging features and the text features using a machine learning based task network. The medical imaging analysis task may comprise any suitable medical imaging analysis task, such as, e.g., segmentation, registration, classification, detection, diagnosis, medical data summarization, etc.

In one embodiment, the machine learning based task network is an LLM based task network. However, the machine learning based task network may be implemented according to any suitable machine learning based architecture. The LLM based task network may be any suitable pre-trained deep learning based LLM, such as, e.g., a transformer-based network (e.g., GPT-based LLMs, BMOOM, BERT). In one embodiment, the LLM based task network is constrained to a specific medical domain (e.g., detecting pneumothorax in chest x-ray images).

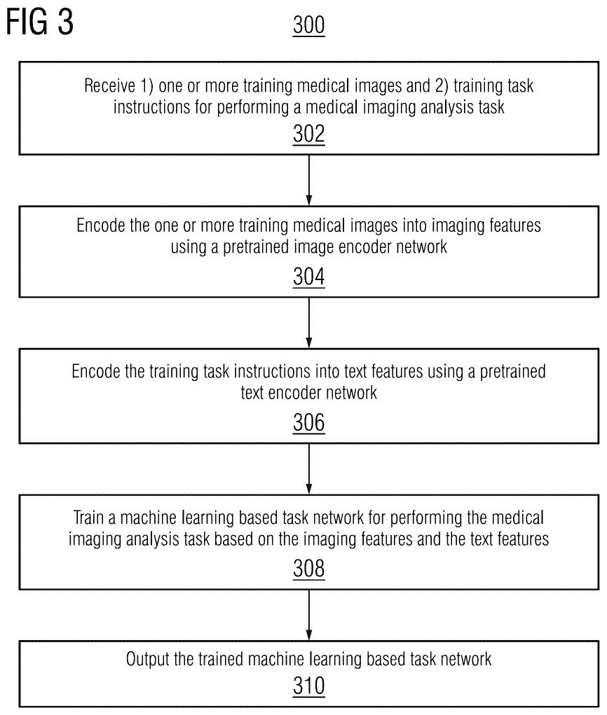

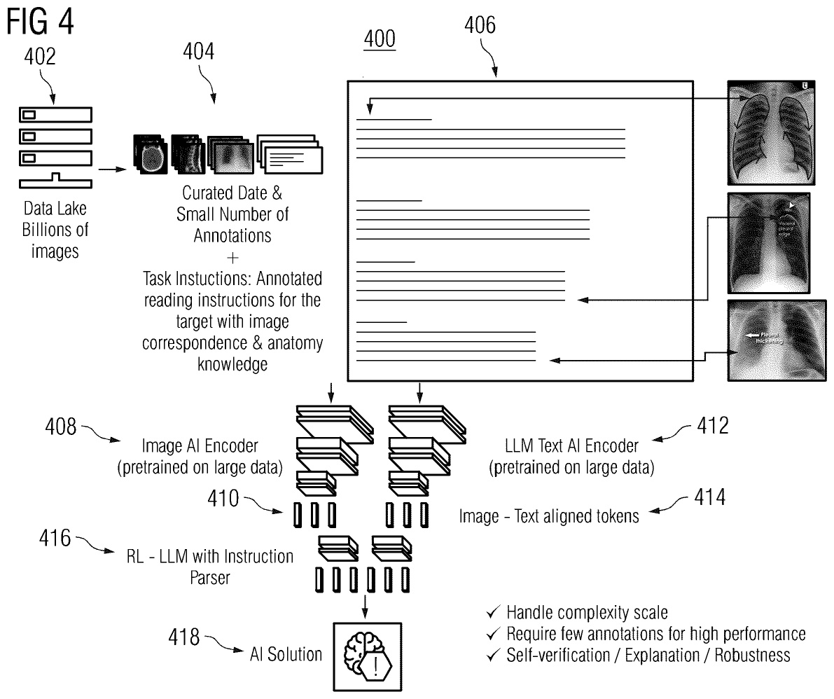

The LLM based task network receives the imaging features and the text features as input (e.g., as one or more prompts) and generates results of the medical imaging analysis task as output. The LLM based task network also receives instructions for performing the medical imaging analysis task. The instructions may be received indirectly through the text encoder networks and/or the image encoder network or directly as separate task instructions. In one embodiment, the LLM based task network may comprise a plurality of heads each for performing a respective medical imaging analysis task (e.g., text decoding, classification score, segmentation, detection, etc.). The LLM based task network is trained during a prior offline or training stage. For example, the LLM based task network may be trained using a relatively small amount of annotated training images and task instructions using few shot learning and self-supervised learning, as described in further detail below with respect to FIGS. 3 and 4. Once trained, the LLM based task network is applied during an online or inference stage, for example, to perform step 108 of FIG. 1.

At step 110 of FIG. 1, results of the medical imaging analysis task are output. For example, the results of the medical imaging analysis task can be output by displaying the results of the medical imaging analysis task on a display device of a computer system, storing the results of the medical imaging analysis task on a memory or storage of a computer system, or by transmitting the results of the medical imaging analysis task to a remote computer system.

In one embodiment, the task instructions (received at step 102 of FIG. 1) may comprise verification steps for the final system to perform (for self-verification) to ensure information consistency for increased operational robustness, which can be combined with well-known uncertainty estimation techniques.

In one embodiment, additional instructions may be received from a user via an interactive user interface. The additional instructions may be parsed and validated based on the one or more input medical images and the current knowledge. The interactive user interface may be utilized for probing the user for explanations for the current medical imaging analysis task. This may be utilized for improved workflow efficiency using LLMs.

FIG. 3 shows a method 300 for training a machine learning based task network for performing a medical imaging analysis task based on training task instructions, in accordance with one or more embodiments. The steps of method 300 may be performed by one or more suitable computing devices, such as, e.g., computer 902 of FIG. 9. FIG. 4 shows a workflow 400 for training a machine learning based task network for performing a medical imaging analysis task based on training task instructions, in accordance with one or more embodiments. FIG. 3 and FIG. 4 will be described together. The steps of method 300 of FIG. 3 and workflow 400 of FIG. 4 are performed during an offline or training stage for training the machine learning based task network. Once trained, the machine learning based task network is applied during an online or inference stage to perform the medical imaging analysis task, e.g., at step 108 of FIG. 1.

At step 302 of FIG. 3, 1) one or more training medical images and 2) training task instructions for performing a medical imaging analysis task are received. In one embodiment, non-imaging training medical data (e.g., text-based medical data) may also be received at step 302. In one example, as shown in workflow 400 of FIG. 4, the one or more training medical images are training medical images 404 curated from data lake 402 and the training task instructions are training task instructions 406.

The one or more training medical images may depict any anatomical object of interest of a patient and may be of any suitable modality or modalities, such as, e.g., CT, MRI, US, x-ray, or any other medical imaging modality or combinations of medical imaging modalities. The one or more training medical images may comprise 2D images and/or 3D volumes. The one or more training medical images may comprise one or more annotated medical images (e.g., annotated by a user) and one or more unannotated medical images.

The training task instructions may comprise anatomical knowledge, task knowledge, and/or any other suitable information that may be utilized for performing the medical imaging analysis task. During the training stage, the training task instructions are associated with the one or more training medical images. For example, the training task instructions may comprise explicit groundings or references to anatomical image regions and/or task specific image regions in at least one of the one or more training medical images. The groundings are used to align embeddings of corresponding image-text concepts in the latent space. The training task instructions may be extracted from textbooks, publications, websites, etc. or may be user-defined.

The one or more training medical images and the training task instructions (and the non-imaging training medical data) can be received by, for example, loading the training medical images, training task instructions, and/or non-imaging training medical data from a storage or memory of one or more computer systems and/or receiving the training medical images, training task instructions, and/or non-imaging training medical data from one or more remote computer systems. In some embodiments, the training medical images may be received directly from an image acquisition device as the medical images are acquired.

At step 304 of FIG. 3, the one or more training medical images are encoded into imaging features using a pretrained image encoder network. The pretrained image encoder network may be, e.g., an autoencoder, a VAE, or may be implemented according to any other suitable machine learning based architecture. The pretrained image encoder network receives the one or more training medical images as input and generates the imaging features as output. The pretrained image encoder network is pretrained during a prior offline or training stage using a large set of training images. Once trained, the pretrained image encoder network is applied during an online or inference stage, for example, to perform step 304 of FIG. 3. In one example, as shown in workflow 400 of FIG. 4, the pretrained image encoder network is image AI (artificial intelligence) encoder 408, which receives training medical images 404 as input and generates image features 410 as output.

At step 306 of FIG. 3, the training task instructions are encoded into text features using a pretrained text encoder network. In one embodiment, where non-imaging training medical data is also received at step 302 of FIG. 3, the non-imaging training medical data (e.g., the text-based medical data) is encoded with the training task instructions into the imaging features using the pretrained image encoder network.

In one embodiment, the text encoder network is an LLM based text encoder network. However, the text encoder network may be implemented according to any suitable machine learning based architecture. In one embodiment, the LLM based text encoder network is constrained to a specific medical domain. The LLM based text encoder network receives the training task instructions (and possibly the non-imaging training medical data) as input and generates the text features as output. Image features 410 and text features 414 are aligned within the latent space. The LLM based text encoder network is pretrained during a prior offline or training stage using a large set of training text-based data. Once trained, the LLM based text encoder network is applied during an online or inference stage, for example, to perform step 306 of FIG. 3.

In one example, as shown in workflow 400 of FIG. 4, the pretrained text encoder network is LLM text AI encoder 412, which receives training task instructions 406 as input and generates text features 414 as output.

At step 308 of FIG. 3, a machine learning based task network is trained for performing the medical imaging analysis task based on the imaging features and the text features. In one embodiment, the machine learning based task network is an LLM based task network. However, the machine learning based task network may be implemented according to any suitable machine learning based architecture. In one embodiment, the LLM based task network is constrained to a specific medical domain (e.g., detecting pneumothorax in chest x-ray images).

The machine learning based task network receives the image features and the text features and generates results of the medical imaging analysis task as output. The machine learning based task network is trained with few shot learning and self-supervised learning.

In few shot learning, the machine learning based task network is trained to accurately perform the medical imaging analysis task using the one or more annotated training images and annotated task descriptions. Few shot learning applies meta-learning, such that the machine learning based task network learns to learn. During the meta-training stage, the machine learning based task network is trained on a relatively few related tasks. During the meta-testing stage, the machine learning based task network can generalize to unseen (but related) tasks.

In self-supervised learning, the machine learning based task network is trained using the one or more unannotated training medical images and text. The aligned image and text features enable the text features to be utilized to generate task instructions and pseudo-labels for the one or more unannotated training medical images and text.

In one embodiment, several output “heads” can be decoded in a generative framework. Example decoding heads can be text decoding, classification score, segmentation, detection, etc. Utilizing the LLM, implicitly the decoding is performed as text. Training for this can be performed by, for example, predicting the same tokens from ground truths sequentially (e.g., with cross entropy loss function) or by reinforcement learning (with/without human feedback) where some text decoding is more preferred than others.

In one example, as shown in workflow 400 of FIG. 4, the machine learning based task network is RL-LLM 416, which receives image features 410 and text features 414 as input and generates results 418 of the medical imaging analysis task as output.

At step 310 of FIG. 3, the trained machine learning based task network is output. For example, the trained machine learning based task network can be output by storing the trained machine learning based task network on a memory or storage of a computer system or by transmitting the trained machine learning based task network to a remote computer system. In one example, the trained machine learning based task network is applied, e.g., to perform step 108 of FIG. 1.

Embodiments described herein are described with respect to the claimed systems as well as with respect to the claimed methods. Features, advantages or alternative embodiments herein can be assigned to the other claimed objects and vice versa. In other words, claims and embodiments for the systems can be improved with features described or claimed in the context of the respective methods. In this case, the functional features of the method are implemented by physical units of the system.

Furthermore, certain embodiments described herein are described with respect to methods and systems utilizing trained machine learning models, as well as with respect to methods and systems for providing trained machine learning models. Features, advantages or alternative embodiments herein can be assigned to the other claimed objects and vice versa. In other words, claims and embodiments for providing trained machine learning models can be improved with features described or claimed in the context of utilizing trained machine learning models, and vice versa. In particular, datasets used in the methods and systems for utilizing trained machine learning models can have the same properties and features as the corresponding datasets used in the methods and systems for providing trained machine learning models, and the trained machine learning models provided by the respective methods and systems can be used in the methods and systems for utilizing the trained machine learning models.

In general, a trained machine learning model mimics cognitive functions that humans associate with other human minds. In particular, by training based on training data the machine learning model is able to adapt to new circumstances and to detect and extrapolate patterns. Another term for “trained machine learning model” is “trained function.”

In general, parameters of a machine learning model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the machine learning models can be adapted iteratively by several steps of training. In particular, within the training a certain cost function can be minimized. In particular, within the training of a neural network the backpropagation algorithm can be used.

In particular, a machine learning model, such as, e.g., the image encoder network utilized at step 104, the text encoder network utilized at step 106, and the machine learning based task network utilized at step 108 of FIG. 1, the pretrained image encoder network utilized at step 304, the pretrained text encoder network utilized at step 306, and the machine learning based task network utilized at step 308 of FIG. 3, and image AI encoder 408, LLM text AI encoder 412, and RL-LLM 416 of FIG. 4, can comprise, for example, a neural network, a support vector machine, a decision tree and/or a Bayesian network, and/or the machine learning model can be based on, for example, k-means clustering, Q-learning, genetic algorithms and/or association rules. In particular, a neural network can be, e.g., a deep neural network, a convolutional neural network or a convolutional deep neural network. Furthermore, a neural network can be, e.g., an adversarial network, a deep adversarial network and/or a generative adversarial network.

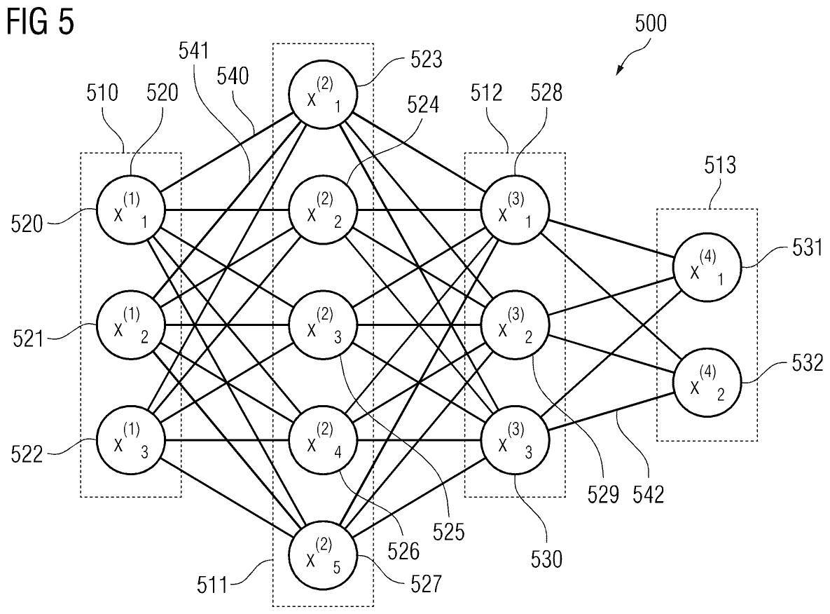

FIG. 5 shows an embodiment of an artificial neural network 500 that may be used to implement one or more machine learning models described herein. Alternative terms for “artificial neural network” are “neural network”, “artificial neural net” or “neural net”.

The artificial neural network 500 comprises nodes 520, . . . , 532 and edges 540, 542, wherein each edge 540, . . . , 542 is a directed connection from a first node 520, 532 to a second node 520, . . . , 532. In general, the first node 520, . . . , 532 and the second node 520, . . . , 532 are different nodes 520, . . . , 532, it is also possible that the first node 520, . . . , 532 and the second node 520, . . . , 532 are identical. For example, in FIG. 5 the edge 540 is a directed connection from the node 520 to the node 523, and the edge 542 is a directed connection from the node 530 to the node 532. An edge 540, . . . , 542 from a first node 520, . . . , 532 to a second node 520, . . . , 532 is also denoted as “ingoing edge” for the second node 520, . . . , 532 and as “outgoing edge” for the first node 520, . . . 532.

In this embodiment, the nodes 520, . . . , 532 of the artificial neural network 500 can be arranged in layers 510, . . . , 513, wherein the layers can comprise an intrinsic order introduced by the edges 540, . . . , 542 between the nodes 520, . . . , 532. In particular, edges 540, . . . , 542 can exist only between neighboring layers of nodes. In the displayed embodiment, there is an input layer 510 comprising only nodes 520, . . . , 522 without an incoming edge, an output layer 513 comprising only nodes 531, 532 without outgoing edges, and hidden layers 511, 512 in-between the input layer 510 and the output layer 513. In general, the number of hidden layers 511, 512 can be chosen arbitrarily. The number of nodes 520, . . . , 522 within the input layer 510 usually relates to the number of input values of the neural network, and the number of nodes 531, 532 within the output layer 513 usually relates to the number of output values of the neural network.

In particular, a (real) number can be assigned as a value to every node 520, . . . , 532 of the neural network 500. Here, x⁽ⁿ⁾ᵢ denotes the value of the i-th node 520, . . . , 532 of the n-th layer 510, . . . , 513. The values of the nodes 520, . . . , 522 of the input layer 510 are equivalent to the input values of the neural network 500, the values of the nodes 531, 532 of the output layer 513 are equivalent to the output value of the neural network 500. Furthermore, each edge 540, . . . , 542 can comprise a weight being a real number, in particular, the weight is a real number within the interval [−1, 1] or within the interval [0, 1]. Here, w^{(m,n)}_{i,j} denotes the weight of the edge between the i-th node 520, . . . , 532 of the m-th layer 510, . . . , 513 and the j-th node 520, . . . , 532 of the n-th layer 510, . . . , 513. Furthermore, the abbreviation w⁽ⁿ⁾_{i,j} is defined for the weight w^{(n,n+1)}_{i,j}.

In particular, to calculate the output values of the neural network 500, the input values are propagated through the neural network. In particular, the values of the nodes 520, . . . , 532 of the (n+1)-th layer 510, . . . , 513 can be calculated based on the values of the nodes 520, . . . , 532 of the n-th layer 510, . . . , 513 by

$${x}^{{( n + 1 )}_{j}} = f ( {\sum }_{i} {x}^{{( n )}_{i}} \cdot {w}^{{( n )}_{i , j}} ) .$$

Herein, the function f is a transfer function (another term is “activation function”). Known transfer functions are step functions, sigmoid function (e.g., the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 510 are given by the input of the neural network 500, wherein values of the first hid-den layer 511 can be calculated based on the values of the input layer 510 of the neural network, wherein values of the second hidden layer 512 can be calculated based in the values of the first hidden layer 511, etc.

In order to set the values w^{(m,n)}^{_{i,j}} for the edges, the neural network 500 has to be trained using training data. In particular, training data comprises training input data and training output data (denoted as tᵢ). For a training step, the neural network 500 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 500 (backpropagation algorithm). In particular, the weights are changed according to

w′⁽ⁿ⁾^{_{i,j}}=w′⁽ⁿ⁾^{_{i,j}}−γ·δ⁽ⁿ⁾^{ⱼ}·x⁽ⁿ⁾^{ᵢ}

wherein γ is a learning rate, and the numbers δ⁽ⁿ⁾ⱼ can be recursively calculated as

$${δ}^{{( n )}_{j}} = ( {\sum }_{k} {δ}^{{( n + 1 )}_{k}} \cdot {w}^{{( n + 1 )}_{j , k}} ) \cdot {f}^{′} ( {\sum }_{i} {x}^{{( n )}_{i}} \cdot {w}^{{( n )}_{i , j}} )$$

based on δ⁽ⁿ⁺¹⁾ⱼ, if the (n+1)-th layer is not the output layer, and

$${δ}^{{( n )}_{j}} = ( {x}^{{( n + 1 )}_{j}} - {t}^{{( n + 1 )}_{j}} ) \cdot {f}^{′} ( {x}^{{( n )}_{i}} \cdot {w}^{{( n )}_{i , j}} )$$

if the (n+1)-th layer is the output layer 513, wherein f′ is the first derivative of the activation function, and t⁽ⁿ⁺¹⁾ⱼ is the comparison training value for the j-th node of the output layer 513.

A convolutional neural network is a neural network that uses a convolution operation instead general matrix multiplication in at least one of its layers (so-called “convolutional layer”). In particular, a convolutional layer performs a dot product of one or more convolution kernels with the convolutional layer's input data/image, wherein the entries of the one or more convolution kernel are the parameters or weights that are adapted by training. In particular, one can use the Frobenius inner product and the ReLU activation function. A convolutional neural network can comprise additional layers, e.g., pooling layers, fully connected layers, and normalization layers.

By using convolutional neural networks input images can be processed in a very efficient way, because a convolution operation based on different kernels can extract various image features, so that by adapting the weights of the convolution kernel the relevant image features can be found during training. Furthermore, based on the weight-sharing in the convolutional kernels less parameters need to be trained, which prevents overfitting in the training phase and allows to have faster training or more layers in the network, improving the performance of the network.

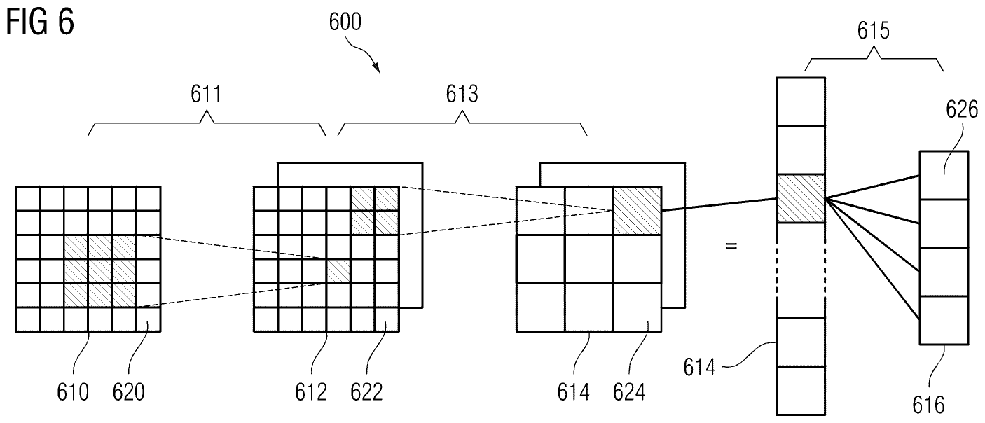

FIG. 6 shows an embodiment of a convolutional neural network 600 that may be used to implement one or more machine learning models described herein. In the displayed embodiment, the convolutional neural network comprises 600 an input node layer 610, a convolutional layer 611, a pooling layer 613, a fully connected layer 614 and an output node layer 616, as well as hidden node layers 612, 614. Alternatively, the convolutional neural network 600 can comprise several convolutional layers 611, several pooling layers 613 and several fully connected layers 615, as well as other types of layers. The order of the layers can be chosen arbitrarily, usually fully connected layers 615 are used as the last layers before the output layer 616.

In particular, within a convolutional neural network 600 nodes 620, 622, 624 of a node layer 610, 612, 614 can be considered to be arranged as a d-dimensional matrix or as a d-dimensional image. In particular, in the two-dimensional case the value of the node 620, 622, 624 indexed with i and j in the n-th node layer 610, 612, 614 can be denoted as x(n)[i,j]. However, the arrangement of the nodes 620, 622, 624 of one node layer 610, 612, 614 does not have an effect on the calculations executed within the convolutional neural network 600 as such, since these are given solely by the structure and the weights of the edges.

A convolutional layer 611 is a connection layer between an anterior node layer 610 (with node values x(n−1)) and a posterior node layer 612 (with node values x(n)). In particular, a convolutional layer 611 is characterized by the structure and the weights of the incoming edges forming a convolution operation based on a certain number of kernels. In particular, the structure and the weights of the edges of the convolutional layer 611 are chosen such that the values x(n) of the nodes 622 of the posterior node layer 612 are calculated as a convolution x(n)=K*x(n−1) based on the values x(n−1) of the nodes 620 anterior node layer 610, where the convolution * is defined in the two-dimensional case as

$${x}_{k}^{( n )} [ i , j ] = ( K * {x}^{( n - 1 )} ) [ i , j ] = {\sum }_{{i}^{′}} {\sum }_{{j}^{′}} K [ {i}^{′} , {j}^{′} ] \cdot {x}^{( n - 1 )} [ i - {i}^{′} , j - {j}^{′} ] .$$

Here the kernel K is a d-dimensional matrix (in this embodiment, a two-dimensional matrix), which is usually small compared to the number of nodes 620, 622 (e.g., a 3×3 matrix, or a 5×5 matrix). In particular, this implies that the weights of the edges in the convolution layer 611 are not independent, but chosen such that they produce said convolution equation. In particular, for a kernel being a 3×3 matrix, there are only 9 independent weights (each entry of the kernel matrix corresponding to one independent weight), irrespectively of the number of nodes 620, 622 in the anterior node layer 610 and the posterior node layer 612.

In general, convolutional neural networks 600 use node layers 610, 612, 614 with a plurality of channels, in particular, due to the use of a plurality of kernels in convolutional layers 611. In those cases, the node layers can be considered as (d+1)-dimensional matrices (the first dimension indexing the channels). The action of a convolutional layer 611 is then a two-dimensional example defined as

$${x}^{{( n )}_{b}} [ i , j ] = {\sum }_{a} {K}_{a , b} * {x}^{{( n - 1 )}_{a}} [ i , j ] = {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ]$$

where x⁽ⁿ⁻¹⁾^{ₐ} corresponds to the a-th channel of the anterior node layer 610, x⁽ⁿ⁾^{_{b}} corresponds to the b-th channel of the posterior node layer 612 and K_{a,b} corresponds to one of the kernels. If a convolutional layer 611 acts on an anterior node layer 610 with A channels and outputs a posterior node layer 612 with B channels, there are A-B independent d-dimensional kernels K_{a,b}.

In general, in convolutional neural networks 600 activation functions are used. In this embodiment re ReLU (acronym for “Rectified Linear Units”) is used, with R(z)=max(0, z), so that the action of the convolutional layer 611 in the two-dimensional example is

$${x}^{{( n )}_{b}} [ i , j ] = R ( {\sum }_{a} ( {K}_{a , b} * {x}^{{( n - 1 )}_{a}} ) [ i , j ] ) = R ( {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ] )$$

It is also possible to use other activation functions, e.g., ELU (acronym for “Exponential Linear Unit”), LeakyReLU, Sigmoid, Tanh or Softmax.

In the displayed embodiment, the input layer 610 comprises 36 nodes 620, arranged as a two-dimensional 6×6 matrix. The first hidden node layer 612 comprises 72 nodes 622, arranged as two two-dimensional 6×6 matrices, each of the two matrices being the result of a convolution of the values of the input layer with a 3×3 kernel within the convolutional layer 611. Equivalently, the nodes 622 of the first hidden node layer 612 can be interpreted as arranged as a three-dimensional 2×6×6 matrix, wherein the first dimension correspond to the channel dimension.

The advantage of using convolutional layers 611 is that spatially local correlation of the input data can exploited by enforcing a local connectivity pattern between nodes of adjacent layers, in particular by each node being connected to only a small region of the nodes of the preceding layer.

A pooling layer 613 is a connection layer between an anterior node layer 612 (with node values x(n−1)) and a posterior node layer 614 (with node values x(n)). In particular, a pooling layer 613 can be characterized by the structure and the weights of the edges and the activation function forming a pooling operation based on a non-linear pooling function f. For example, in the two-dimensional case the values x(n) of the nodes 624 of the posterior node layer 614 can be calculated based on the values x(n−1) of the nodes 622 of the anterior node layer 612 as

$${x}^{{( n )}_{b}} [ i , j ] = f ( {x}^{( n - 1 )} [ {id}_{1} , {jd}_{2} ] , ... , {x}^{{( n - 1 )}_{b}} [ ( i + 1 ) {d}_{1} - 1 , ( j + 1 ) {d}_{2} - 1 ] )$$

In other words, by using a pooling layer 613 the number of nodes 622, 624 can be reduced, by re-placing a number d1-d2 of neighboring nodes 622 in the anterior node layer 612 with a single node 622 in the posterior node layer 614 being calculated as a function of the values of said number of neighboring nodes. In particular, the pooling function f can be the max-function, the average or the L2-Norm. In particular, for a pooling layer 613 the weights of the incoming edges are fixed and are not modified by training.

The advantage of using a pooling layer 613 is that the number of nodes 622, 624 and the number of parameters is reduced. This leads to the amount of computation in the network being reduced and to a control of overfitting.

In the displayed embodiment, the pooling layer 613 is a max-pooling layer, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

In general, the last layers of a convolutional neural network 600 are fully connected layers 615. A fully connected layer 615 is a connection layer between an anterior node layer 614 and a posterior node layer 616. A fully connected layer 613 can be characterized by the fact that a majority, in particular, all edges between nodes 614 of the anterior node layer 614 and the nodes 616 of the posterior node layer are present, and wherein the weight of each of these edges can be adjusted individually.

In this embodiment, the nodes 624 of the anterior node layer 614 of the fully connected layer 615 are displayed both as two-dimensional matrices, and additionally as non-related nodes (indicated as a line of nodes, wherein the number of nodes was reduced for a better presentability). This operation is also denoted as “flattening”. In this embodiment, the number of nodes 626 in the posterior node layer 616 of the fully connected layer 615 smaller than the number of nodes 624 in the anterior node layer 614. Alternatively, the number of nodes 626 can be equal or larger.

Furthermore, in this embodiment the Softmax activation function is used within the fully connected layer 615. By applying the Softmax function, the sum the values of all nodes 626 of the output layer 616 is 1, and all values of all nodes 626 of the output layer 616 are real numbers between 0 and 1. In particular, if using the convolutional neural network 600 for categorizing input data, the values of the output layer 616 can be interpreted as the probability of the input data falling into one of the different categories.

In particular, convolutional neural networks 600 can be trained based on the backpropagation algorithm. For preventing overfitting, methods of regularization can be used, e.g., dropout of nodes 620, . . . , 624, stochastic pooling, use of artificial data, weight decay based on the L1 or the L2 norm, or max norm constraints.

According to an aspect, the machine learning model may comprise one or more residual networks (ResNet). In particular, a ResNet is an artificial neural network comprising at least one jump or skip connection used to jump over at least one layer of the artificial neural network. In particular, a ResNet may be a convolutional neural network comprising one or more skip connections respectively skipping one or more convolutional layers. According to some examples, the ResNets may be represented as m-layer ResNets, where m is the number of layers in the corresponding architecture and, according to some examples, may take values of 34, 50, 101, or 152. According to some examples, such an m-layer ResNet may respectively comprise (m−2)/2 skip connections.

A skip connection may be seen as a bypass which directly feeds the output of one preceding layer over one or more bypassed layers to a layer succeeding the one or more bypassed layers. Instead of having to directly fit a desired mapping, the bypassed layers would then have to fit a residual mapping “balancing” the directly fed output.

Fitting the residual mapping is computationally easier to optimize than the directed mapping. What is more, this alleviates the problem of vanishing/exploding gradients during optimization upon training the machine learning models: if a bypassed layer runs into such problems, its contribution may be skipped by regularization of the directly fed output. Using ResNets thus brings about the advantage that much deeper networks may be trained.

In particular, a recurrent machine learning model is a machine learning model whose output does not only depend on the input value and the parameters of the machine learning model adapted by the training process, but also on a hidden state vector, wherein the hidden state vector is based on previous inputs used on for the recurrent machine learning model. In particular, the recurrent machine learning model can comprise additional storage states or additional structures that incorporate time delays or comprise feedback loops.

In particular, the underlying structure of a recurrent machine learning model can be a neural network, which can be denoted as recurrent neural network. Such a recurrent neural network can be described as an artificial neural network where connections between nodes form a directed graph along a temporal sequence. In particular, a recurrent neural network can be interpreted as directed acyclic graph. In particular, the recurrent neural network can be a finite impulse recurrent neural network or an infinite impulse recurrent neural network (wherein a finite impulse network can be unrolled and replaced with a strictly feedforward neural network, and an infinite impulse network cannot be unrolled and replaced with a strictly feedforward neural network).

In particular, training a recurrent neural network can be based on the BPTT algorithm (acronym for “backpropagation through time”), on the RTRL algorithm (acronym for “real-time recurrent learning”) and/or on genetic algorithms.

By using a recurrent machine learning model input data comprising sequences of variable length can be used. In particular, this implies that the method cannot be used only for a fixed number of input datasets (and needs to be trained differently for every other number of input datasets used as input), but can be used for an arbitrary number of input datasets. This implies that the whole set of training data, independent of the number of input datasets contained in different sequences, can be used within the training, and that training data is not reduced to training data corresponding to a certain number of successive input datasets.

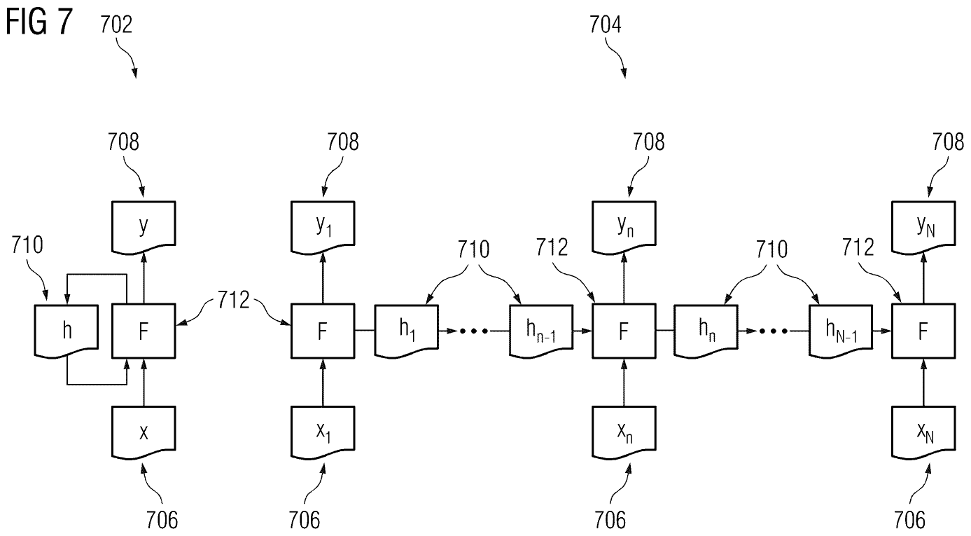

FIG. 7 shows a schematic structure of a recurrent machine learning model F, both in a recurrent representation 702 and in an unfolded representation 704, that may be used to implement one or more machine learning models described herein. The recurrent machine learning model takes as input several input datasets x, x₁, . . . , x_{N} 706 and creates a corresponding set of output datasets y, y₁, . . . , y_{N} 708. Furthermore, the output depends on a so-called hidden vector h, h₁, . . . , h_{N} 710, which implicitly comprises information about input datasets previously used as input for the recurrent machine learning model F 712. By using these hidden vectors h, h₁, . . . , h_{N} 710, a sequentiality of the input datasets can be leveraged.

In a single step of the processing, the recurrent machine learning model F 712 takes as input the hidden vector hₙ₋₁ created within the previous step and an input dataset xₙ. Within this step, the recurrent machine learning model F generates as output an updated hidden vector hₙ and an output dataset yₙ. In other words, one step of processing calculates (yₙ, hₙ)=F(xₙ, hₙ₋₁), or by splitting the recurrent machine learning model F 712 into a part F(y) calculating the output data and F(h) calculating the hidden vector, one step of processing calculates yₙ=F^{(y)}(xₙ, hₙ₋₁) and hₙ=F^{(h)}(xₙ, hₙ₋₁). For the first processing step, h₀ can be chosen randomly or filled with all entries being zero. The parameters of the recurrent machine learning model F 712 that were trained based on training datasets before do not change between the different processing steps.

In particular, the output data and the hidden vector of a processing step depend on all the previous input datasets used in the previous steps. yₙ=F^{(y)}(xₙ, F^{(h)}(xₙ₋₁, hₙ₋₂)) and hₙ=F(h)(xₙ, F^{(h)}(xₙ₋₁, hₙ₋₂)).

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatuses, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatuses, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIG. 1, 3, or 4. Certain steps or functions of the methods and workflows described herein, including one or more of the steps or functions of FIG. 1, 3, or 4, may be performed by a server or by another processor in a network-based cloud-computing system. Certain steps or functions of the methods and workflows described herein, including one or more of the steps of FIG. 1, 3, or 4, may be performed by a client computer in a network-based cloud computing system. The steps or functions of the methods and workflows described herein, including one or more of the steps of FIG. 1, 3, or 4, may be performed by a server and/or by a client computer in a network-based cloud computing system, in any combination.

Systems, apparatuses, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIG. 1, 3, or 4, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

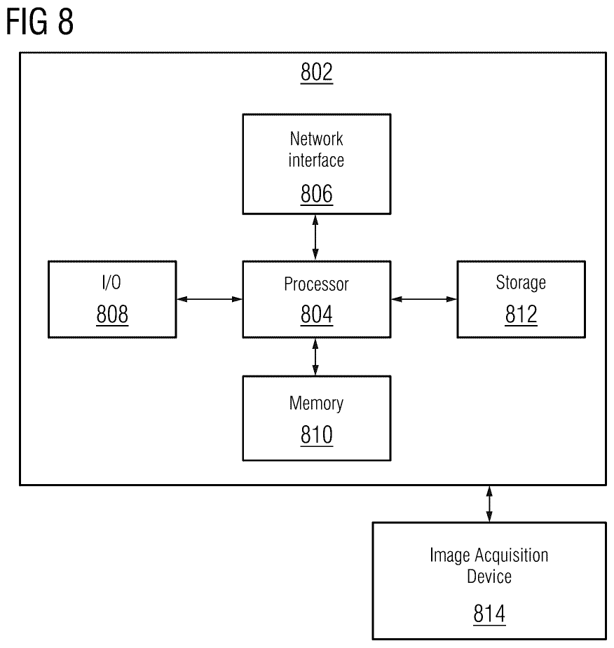

A high-level block diagram of an example computer 802 that may be used to implement systems, apparatuses, and methods described herein is depicted in FIG. 8. Computer 802 includes a processor 804 operatively coupled to a data storage device 812 and a memory 810. Processor 804 controls the overall operation of computer 802 by executing computer program instructions that define such operations. The computer program instructions may be stored in data storage device 812, or other computer readable medium, and loaded into memory 810 when execution of the computer program instructions is desired. Thus, the method and workflow steps or functions of FIG. 1, 3, or 4 can be defined by the computer program instructions stored in memory 810 and/or data storage device 812 and controlled by processor 804 executing the computer program instructions. For example, the computer program instructions can be implemented as computer executable code programmed by one skilled in the art to perform the method and workflow steps or functions of FIG. 1, 3, or 4. Accordingly, by executing the computer program instructions, the processor 804 executes the method and workflow steps or functions of FIG. 1, 3, or 4. Computer 802 may also include one or more network interfaces 806 for communicating with other devices via a network. Computer 802 may also include one or more input/output devices 808 that enable user interaction with computer 802 (e.g., display, keyboard, mouse, speakers, buttons, etc.).

Processor 804 may include both general and special purpose microprocessors, and may be the sole processor or one of multiple processors of computer 802. Processor 804 may include one or more central processing units (CPUs), for example. Processor 804, data storage device 812, and/or memory 810 may include, be supplemented by, or incorporated in, one or more application-specific integrated circuits (ASICs) and/or one or more field programmable gate arrays (FPGAs).

Data storage device 812 and memory 810 each include a tangible non-transitory computer readable storage medium. Data storage device 812, and memory 810, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 808 may include peripherals, such as a printer, scanner, display screen, etc. For example, input/output devices 808 may include a display device such as a cathode ray tube (CRT) or liquid crystal display (LCD) monitor for displaying information to the user, a keyboard, and a pointing device such as a mouse or a trackball by which the user can provide input to computer 802.

An image acquisition device 814 can be connected to the computer 802 to input image data (e.g., medical images) to the computer 802. It is possible to implement the image acquisition device 814 and the computer 802 as one device. It is also possible that the image acquisition device 814 and the computer 802 communicate wirelessly through a network. In a possible embodiment, the computer 802 can be located remotely with respect to the image acquisition device 814.

Any or all of the systems, apparatuses, and methods discussed herein may be implemented using one or more computers such as computer 802.

One skilled in the art will recognize that an implementation of an actual computer or computer system may have other structures and may contain other components as well, and that FIG. 8 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

The following is a list of non-limiting illustrative embodiments disclosed herein:

Illustrative embodiment 1. A computer-implemented method comprising: receiving 1) one or more input medical images of a patient and 2) task instructions for performing a medical imaging analysis task; encoding the one or more input medical images into imaging features using an image encoder network; encoding the task instructions into text features using a text encoder network; performing the medical imaging analysis task based on the imaging features and the text features using a machine learning based task network; and outputting results of the medical imaging analysis task.

Illustrative embodiment 2. The computer-implemented method of illustrative embodiment 1, wherein the task instructions comprise references to image regions in at least one of the one or more input medical images.

Illustrative embodiment 3. The computer-implemented method according to one of the preceding embodiments, wherein the task instructions comprise anatomical knowledge and task knowledge. The task knowledge comprises at least one of a description of an anatomical abnormality, how the anatomical abnormality is represented, and how the anatomical abnormality can be detected in the one or more input medical images.

Illustrative embodiment 4. The computer-implemented method according to one of the preceding embodiments, wherein the task instructions are user-defined.

Illustrative embodiment 5. The computer-implemented method according to one of the preceding embodiments, wherein the text features and the imaging features are aligned in a same latent space.

Illustrative embodiment 6. The computer-implemented method according to one of the preceding embodiments, wherein the machine learning based task network is trained with self-supervised learning based on unannotated training medical images and text.

Illustrative embodiment 7. The computer-implemented method according to one of the preceding embodiments, wherein the machine learning based task network is trained with few shot learning using annotated training medical images and annotated task descriptions.

Illustrative embodiment 8. The computer-implemented method according to one of the preceding embodiments, wherein the machine learning based task network comprises an LLM (large language model) based task network.

Illustrative embodiment 9. The computer-implemented method according to one of the preceding embodiments, wherein: receiving 1) one or more input medical images of a patient and 2) task instructions for performing a medical imaging analysis task comprises receiving text-based medical data of the patient; and encoding the task instructions into text features using a text encoder network comprises encoding the task instructions and the text-based medical data into the text features using the text encoder network.

Illustrative embodiment 10. An apparatus comprising: means for receiving 1) one or more input medical images of a patient and 2) task instructions for performing a medical imaging analysis task; means for encoding the one or more input medical images into imaging features using an image encoder network; means for encoding the task instructions into text features using a text encoder network; means for performing the medical imaging analysis task based on the imaging features and the text features using a machine learning based task network; and means for outputting results of the medical imaging analysis task.

Illustrative embodiment 11. The apparatus of illustrative embodiment 10, wherein the task instructions comprise references to image regions in at least one of the one or more input medical images.

Illustrative embodiment 12. The apparatus according to one of illustrative embodiments 10-11, wherein the task instructions comprise anatomical knowledge and task knowledge. The task knowledge comprises at least one of a description of an anatomical abnormality, how the anatomical abnormality is represented, and how the anatomical abnormality can be detected in the one or more input medical images.

Illustrative embodiment 13. The apparatus according to one of illustrative embodiments 10-12, wherein the task instructions are user-defined.

Illustrative embodiment 14. A non-transitory computer-readable medium comprising instructions which, when executed by a computer, cause the computer to carry out operations comprising: receiving 1) one or more input medical images of a patient and 2) task instructions for performing a medical imaging analysis task; encoding the one or more input medical images into imaging features using an image encoder network; encoding the task instructions into text features using a text encoder network; performing the medical imaging analysis task based on the imaging features and the text features using a machine learning based task network; and outputting results of the medical imaging analysis task.

Illustrative embodiment 15. The non-transitory computer-readable medium of illustrative embodiment 14, wherein the text features and the imaging features are aligned in a same latent space.

Illustrative embodiment 16. The non-transitory computer-readable medium according to one of illustrative embodiments 14-15, wherein the machine learning based task network is trained with self-supervised learning based on unannotated training medical images and text.

Illustrative embodiment 17. The non-transitory computer-readable medium according to one of illustrative embodiments 14-16, wherein the machine learning based task network is trained with few shot learning using annotated training medical images and annotated task descriptions.

Illustrative embodiment 18. A computer-implemented method comprising: receiving 1) one or more training medical images and 2) training task instructions for performing a medical imaging analysis task; encoding the one or more training medical images into imaging features using a pretrained image encoder network; encoding the training task instructions into text features using a pretrained text encoder network; training a machine learning based task network for performing the medical imaging analysis task based on the imaging features and the text features; and outputting the trained machine learning based task network.

Illustrative embodiment 19. The computer-implemented method of illustrative embodiment 18, wherein the machine learning based task network is trained with self-supervised learning based on unannotated training medical images and text.

Illustrative embodiment 20. The computer-implemented method according to one of illustrative embodiments 18-19, wherein the machine learning based task network is trained with few shot learning using annotated training medical images and annotated task descriptions.

1. A computer-implemented method comprising:
receiving 1) one or more input medical images of a patient and 2) task instructions for performing a medical imaging analysis task;
encoding the one or more input medical images into imaging features using an image encoder network;
encoding the task instructions into text features using a text encoder network;
performing the medical imaging analysis task based on the imaging features and the text features using a machine learning based task network; and
outputting results of the medical imaging analysis task.

2. The computer-implemented method of claim 1, wherein the task instructions comprise references to image regions in at least one of the one or more input medical images.

3. The computer-implemented method of claim 1, wherein the task instructions comprise anatomical knowledge and task knowledge, the task knowledge comprising at least one of a description of an anatomical abnormality, how the anatomical abnormality is represented, and how the anatomical abnormality can be detected in the one or more input medical images.

4. The computer-implemented method of claim 1, wherein the task instructions are user-defined.

5. The computer-implemented method of claim 1, wherein the text features and the imaging features are aligned in a same latent space.

6. The computer-implemented method of claim 1, wherein the machine learning based task network is trained with self-supervised learning based on unannotated training medical images and text.

7. The computer-implemented method of claim 1, wherein the machine learning based task network is trained with few shot learning using annotated training medical images and annotated task descriptions.

8. The computer-implemented method of claim 1, wherein the machine learning based task network comprises an LLM (large language model) based task network.

9. The computer-implemented method of claim 1, wherein:
receiving 1) one or more input medical images of a patient and 2) task instructions for performing a medical imaging analysis task comprises receiving text-based medical data of the patient; and
encoding the task instructions into text features using a text encoder network comprises encoding the task instructions and the text-based medical data into the text features using the text encoder network.

10. An apparatus comprising:
means for receiving 1) one or more input medical images of a patient and 2) task instructions for performing a medical imaging analysis task;
means for encoding the one or more input medical images into imaging features using an image encoder network;
means for encoding the task instructions into text features using a text encoder network;
means for performing the medical imaging analysis task based on the imaging features and the text features using a machine learning based task network; and
means for outputting results of the medical imaging analysis task.

11. The apparatus of claim 10, wherein the task instructions comprise references to image regions in at least one of the one or more input medical images.

12. The apparatus of claim 10, wherein the task instructions comprise anatomical knowledge and task knowledge, the task knowledge comprising at least one of a description of an anatomical abnormality, how the anatomical abnormality is represented, and how the anatomical abnormality can be detected in the one or more input medical images.

13. The apparatus of claim 10, wherein the task instructions are user-defined.

14. A non-transitory computer-readable medium comprising instructions which, when executed by a computer, cause the computer to carry out operations comprising:
receiving 1) one or more input medical images of a patient and 2) task instructions for performing a medical imaging analysis task;
encoding the one or more input medical images into imaging features using an image encoder network;
encoding the task instructions into text features using a text encoder network;
performing the medical imaging analysis task based on the imaging features and the text features using a machine learning based task network; and
outputting results of the medical imaging analysis task.

15. The non-transitory computer-readable medium of claim 14, wherein the text features and the imaging features are aligned in a same latent space.

16. The non-transitory computer-readable medium of claim 14, wherein the machine learning based task network is trained with self-supervised learning based on unannotated training medical images and text.

17. The non-transitory computer-readable medium of claim 14, wherein the machine learning based task network is trained with few shot learning using annotated training medical images and annotated task descriptions.

18. A computer-implemented method comprising:
receiving 1) one or more training medical images and 2) training task instructions for performing a medical imaging analysis task;
encoding the one or more training medical images into imaging features using a pretrained image encoder network;
encoding the training task instructions into text features using a pretrained text encoder network;
training a machine learning based task network for performing the medical imaging analysis task based on the imaging features and the text features; and
outputting the trained machine learning based task network.

19. The computer-implemented method of claim 18, wherein the machine learning based task network is trained with self-supervised learning based on unannotated training medical images and text.

20. The computer-implemented method of claim 18, wherein the machine learning based task network is trained with few shot learning using annotated training medical images and annotated task descriptions.
